(19) United States
(12) Patent Application Publication
(10) Pub. No.: US 2009/0227606 A1
(43) Pub. Date: Sep. 10, 2009

(54) Methods for Preventing or Treating Acute and Chronic Pain

(76) Inventor: Joyce A. DeLeo, Lebanon, NH (US)

Correspondence Address: LICATA & TYRRELL P.C., 66 E. MAIN STREET, MARLTON, NJ, 08053, US

(21) Appl. No.: 12/301,246

(22) PCT Filed: May 21, 2007
(86) PCT No.: PCT/US07/69328

§ 371 Date: Nov. 18, 2008

Related U.S. Application Data
(60) Provisional application No. 60/802,043, filed on May 19, 2006.
(60) Provisional application No. 60/807,646, filed on Jul. 18, 2006.

(51) Int. Cl. A61K 31/522; C12Q 1/02; G01N 33/68; A61P 29/00

(52) U.S. Cl. 514/263.34; 435/29; 436/86

(57) ABSTRACT

The present invention is a method for preventing or treating acute and chronic pain. Using an agent to increase the expression or activity of GLT-1, behavioral hypersensitivity is attenuated thereby preventing or treating acute and chronic pain in a subject in need of such treatment. Methods for identifying agents that prevent or treat acute and chronic pain through modulation of the expression or activity of GLT-1 or the GLT-1 pathway are also provided.

### INTRODUCTION

This invention was made in the course of research sponsored in part by National Institute of Drug Abuse (NIDA) grant DA11276. The U.S. government may have certain rights in this invention.

### BACKGROUND OF THE INVENTION

Neuropathic pain is a debilitating condition that affects millions of individuals worldwide. Unfortunately, treatment options for these patients are limited as opioids and other available pharmacotherapies, such as antiepileptic drugs and non-steroidal anti-inflammatory drugs (NSAIDs), are not able to provide long term relief of associated spontaneous pain, allodynia and hyperalgesia in many patients. In an effort to uncover mechanisms that could lead to novel drug targets, one focus has been on the role of spinal neuroimmune activation (glial activation and immune mediator expression) in nerve injury-induced behavioral sensitization, where it has been demonstrated that peripheral nerve damage induces activation of spinal astrocytes and microglia as well as enhancement of cytokine expression which correlates with behavioral hypersensitivity (Colburn, R. W. et al. 1999. Exp. Neurol. 157:289-304; DeLeo, J. A. et al. 1997. Brain Res. 759:50-57).

Glutamate is the primary excitatory neurotransmitter in the central nervous system (CNS) and as such, regulation of its synaptic concentration by high affinity transporters is crucial to avoid excitotoxicity (Danbolt, N. C. 2001. Prog. Neurobiol. 65:1-105). Mature, differentiated astrocytes are known to express the GLT-1 (EAAT2) transporter, which is responsible for over 90% of synaptic glutamate clearance (Tanaka, K. et al. 1997. Science 276:1699-1702). Thus, GLT-1 is considered to be the main player in maintenance of glutamate homeostasis. Interestingly, activated astrocytes are less efficient at clearing glutamate due to a morphological change characterized by a “de-differentiated” phenotype, expressing low levels of GLT-1, high levels of GLAST (a secondary transporter) and high levels of cytokines, chemokines and other pro-inflammatory mediators (Schlag, B. D. et al. 1998. Mol. Pharmacol. 53:355-369; Sweitzer, S. et al. 2001. Neuroscience 103:529-539). It then follows that aberrant astrocytic activation leaves neurons susceptible to enhanced glutamate levels.

The contribution of activated glia to nerve injury-induced mechanical allodynia has been elucidated using propentofylline, an atypical methylxanthine derivative previously shown to attenuate astrocytic activation in a rodent model of ischemia (DeLeo, J. et al. 1987. J. Cereb. Blood Flow Metab 7:745-751). Furthermore, systemically or intrathecally administered propentofylline attenuated mechanical allodynia induced by L5 spinal nerve transection; this effect correlated temporally with a reduction in glial activation (Sweitzer, S. M. et al. 2001. J. Pharmacol. Exp. Ther. 297:1210-1217). Although the specific mechanism of propentofylline-induced anti-allodynia remains unknown, several actions have been proposed. Propentofylline has been shown to inhibit adenosine transport and the cyclic-adenosine-5′,3′-monophosphate (cAMP)-specific phosphodiesterase (PDE IV) leading to the induction of cAMP (Meskini, N. et al. 1994. Biochem. Pharmacol. 47:781-788; Nagata, K. et al. 1985. Arzneimit. Forsch. 35:1034-1036; Parkinson, F. E. and B. B. Fredholm. 1991. Eur. J. Pharmacol. 202:361-366). Enhancement of cAMP (by the non-hydrolysable compound dibutyryl-cAMP) has been previously shown to induce astrocytic maturation and GLT-1 induction in cultured astrocytes (Schlag, B. D. et al. 1998. Mol. Pharmacol. 53:355-369; Zelenaia, O. et al. 2000. Mol. Pharmacol. 57:667-678). Additionally, strengthening of cAMP-dependent signaling decreases microglial proliferation and activation in culture (Si, Q. S. et al. 1996. Exp. Neurol. 137:345-349), providing a possible mechanism for propentofylline-induced glial modulation.

It has now been found that propentofylline acts by very specific mechanisms to affect pain mediators, including differentiation of astrocytes to a homeostatic, mature phenotype that is capable of glutamate clearance and alteration of glial glutamate transporters as a way to control aberrant glial activation, both mechanisms related to control of acute and chronic pain, e.g., neuropathic pain or inflammatory pain.

### SUMMARY OF THE INVENTION

The present invention is a method for preventing or treating acute and chronic pain in a patient which comprises administering to a patient in need of treatment an effective amount of an agent that increases levels of glutamate transporter GLT-1, thereby preventing or treating acute and chronic pain in the patient. In further embodiments the effective amount of the agent administered is in the range of 10 to 100 mg/m². In a preferred embodiment the method of the present invention involves administration of propentofylline or a methylxanthine derivative and the acute and chronic pain being treated is neuropathic pain or inflammatory pain. The method contemplates administration of the agent by a variety of means including orally, sublingually, subcutaneously, intramuscularly, intravenously, or transmucosally.

Another object of the present invention is a method for identifying an agent useful in preventing or treating acute and chronic pain which comprises contacting GLT-1 or a cell expressing GLT-1 with a test agent and measuring the activity or expression level of GLT-1 in the presence and absence of the test agent, wherein an increase in the measured activity or expression level of GLT-1 in the presence of the test agent as compared with the measured activity or expression level of GLT-1 in the absence of the test agent identifies an agent that prevents or treats acute and chronic pain. In a preferred embodiment, the acute and chronic pain being prevented or treated is neuropathic pain or inflammatory pain.

Another object of the present invention is an agent for prevention or treatment of acute and chronic pain, wherein the agent is identified by the method of the present invention

### BRIEF DESCRIPTION OF THE DRAWINGS

FIG. 1 depicts the results of quantitation of cell process length after phase contrast examination of astrocyte cultures and propentofylline-induced astrocyte differentiation at 7 days. The bar graph shows the results demonstrating that db-cAMP induced significantly longer processes than propentofylline (***P<0.001 vs. control or db-cAMP as indicated; the number of astrocyte processes quantified is indicated in each bar). PPF10, 100, 1000: Propentofylline-treated 10, 100 or 1000 μM; db: 250 μM db-cAMP; db-PPF: 250 μM db-cAMP plus 1000 μM propentofylline.

FIG. 2 depicts results of experiments examining transcriptional regulation of glutamate transporters by propentofylline. Real Time RT-PCR clearly demonstrated an enhancement of GLT-1 and GLAST mRNA levels in a dose-dependent manner by propentofylline, also mimicked by db-cAMP. Results are relative to GAP-DH control. PPF10, 100, 1000: Propentofylline-treated 10, 100 or 1000 μM; db-cAMP: 250 μM db-cAMP; db-PPF: 250 μM db-cAMP plus 1000 μM propentofylline. Data are expressed as the means±SEM of 3 independent experiments conducted in duplicate (**P<0.01; ***P<0.001 vs. control).

FIG. 3 depicts the effects of propentofylline treatment on protein expression in cultured astrocytes. Propentofylline dose-dependently increased levels of GLT-1 protein in cultured astrocytes. Astrocyte-enriched cultures were grown until confluent (12-14 days in vitro) and then treated with propentofylline, db-cAMP or both for 3 or 7 days. Equal amounts of protein were loaded (40 μg) in each lane and normalized to β-actin. FIG. 3A shows a representative western blot, demonstrating a significant increase in GLT-1 after propentofylline treatment. FIG. 3B shows the expression levels of GLT-1 relative to control cultures. Data are expressed as the mean±SEM of 3 independent experiments conducted in duplicate (***P<0.001 vs. control).

FIG. 4 depicts the effects of propentofylline on protein expression in cultured astrocytes. Propentofylline dose-dependently increased levels of GLAST protein in cultured astrocytes. Astrocyte-enriched cultures were grown until confluent (12-14 days in vitro) and then treated with propentofylline, db-cAMP or both for 3 or 7 days. Equal amounts of protein were loaded (40 μg) in each lane and normalized to β-actin. FIG. 4A shows a representative western blot, demonstrating a significant increase in GLAST after propentofylline treatment. FIG. 4B shows the expression levels of GLAST relative to control cultures. Data are expressed as the mean±SEM of 3 independent experiments conducted in duplicate (*P<0.05; ***P<0.001 vs. control).

FIG. 5 depicts the effect of propentofylline on glutamate uptake in cultured astrocytes. Propentofylline treatment resulted in a dose-dependent increase in glutamate uptake in cultured astrocytes that was GLT-1 mediated. FIG. 5A shows Na⁺-dependent glutamate transport in vitro using ³H-glutamate in control and 7-day treated cultures. FIG. 5B shows the sensitivity of transport to inhibition by dihydrokainate (DHK), a GLT-1 selective inhibitor. Astrocytes were incubated for 10 minutes in 100 μM DHK prior to substrate addition. Uptake in nmol/mg protein/min was calculated and set relative to control cultures. Data are expressed as relative uptake and are the mean±SEM of 3 independent experiments (*P<0.05; ***P<0.001 vs. control or PPF1000, as indicated).

FIG. 6 depicts the time course of cAMP/db-cAMP induction by propentofylline and db-cAMP. Astrocytes were treated with propentofylline or db-cAMP with the doses shown for 3 or 7 days. As expected, db-cAMP was detected at both time points; in contrast, higher doses of propentofylline did not lead to elevation in cAMP at 3 or 7 days suggesting a transient, rather than sustained effect (***P<0.001 vs. PPF1000. PPF10, 100, 1000: Propentofylline-treated 10, 100 or 1000 μM; db-cAMP: 250 μM db-cAMP; db-PPF: 250 μM db-cAMP plus 1000 μM propentofylline).

FIG. 7 depicts results of experiments examining the effects of propentofylline on cytokine and chemokine release in cultured astrocytes. Propentofylline reversed LPs-induced cytokine and chemokine release from cultured astrocytes. Astrocytes were treated with saline, propentofylline (1000 μM), db-cAMP (250 μM) and/or LPS (1 μg/ml) for seven days. Cell culture supernatant was collected and ELISAs for MIP-2 (FIG. 7A) and MCP-1 (FIG. 7B) were carried out. LPS induced significant chemokine release that was reversed by co-treatment with propentofylline (^{#}P<0.001 vs. LPS alone). Data are expressed as the mean±SEM of 2 independent experiments conducted in duplicate (*P<0.05; **P<0.01; ***P<0.001 vs. control).

FIG. 8 depicts the effects of propentofylline on mechanical allodynia in a rat model of neuropathic pain. Propentofylline attenuates mechanical allodynia in L5 spinal nerve transected rats. Sham and L5 spinal nerve transected rats (L5) received daily injections of 10 μg propentofylline (PPF) or saline via lumbar puncture, beginning one-hour prior to surgery. Preventative treatment with propentofylline resulted in a significant decrease in mechanical allodynia to a 12-g von Frey filament compared with L5 spinal nerve transected, saline (L5 saline) controls (^{#}P<0.001). Mechanical allodynia is reported as the average number of paw withdrawals out of 30±S.E.M. (n=6-8/treatment). Day 0 represents pre-injury responses (***P <0.001 vs. Sham saline).

FIG. 9 depicts the effect of propentofylline on GLT-1 and GLAST mRNA. Real Time RT-PCR was carried out on mRNA obtained from ipsilateral lumbar spinal cord. FIG. 9A shows that GLT-1 mRNA is enhanced by propentofylline (L5, PPF) at days 4 and 12 post-transection. FIG. 9B shows that GLAST mRNA levels are unaffected by propentofylline treatment. The mRNA levels were normalized to the corresponding GAPDH (housekeeping gene) and values shown are mean±S.E.M. (n=4/group; **P<001 vs. sham; *P<0.05, ^{#}P<0.001 vs. L5).

FIG. 10 depicts the effects of propentofylline treatment on GLT-1 protein expression. Treatment with propentofylline leads to a significant increase in GLT-1 protein expression. Western blot analysis was carried out on protein lysates obtained from ipsilateral lumbar spinal cord on days 4 and 12 post-transection, and GLT-1 immunoreactivity was assessed. FIG. 10A is a representative Western blot of GLT-1 and β-actin control. FIG. 10B is a densitometric analysis of blots (n=4/group). At day 12 post-injury, L5 spinal nerve transected rats displayed decreased GLT-1 expression which was reversed with propentofylline treatment (*P<0.05 vs. L5; S: sham, L5: L5 spinal nerve transected, P: L5 spinal nerve transected, propentofylline). Data are represented as the mean±S.E.M. Each western blot was repeated at least 3 times.

FIG. 11 depicts the effects of propentofylline treatment on GLAST expression. Treatment with propentofylline leads to a decrease in GLAST protein expression. Western blot analysis was carried out on protein lysates obtained from ipsilateral lumbar spinal cord on days 4 and 12 post-transection, and GLAST immunoreactivity was assessed. FIG. 11A is a representative Western blot of GLAST and β-actin control. FIG. 11B is a densitometric analysis of blots (n=4/group). At day 4 post-transection, GLAST expression was significantly increased above sham (**P<0.01) while propentofylline treatment maintained GLAST at sham levels (#p<0.001 vs. L5). At day 12 post-injury, L5 spinal nerve transected rats displayed decreased GLAST expression which was further decreased by propentofylline treatment (*P<0.05 vs. sham; S: sham, L5: L5 spinal nerve transected, P: L5 spinal nerve transected, propentofylline). Data are represented as the mean±S.E.M. Each western blot was repeated at least 3 times.

FIG. 12 depicts the anti-allodynic effects of propentofylline treatment in vivo in a transgenic mouse model. Sham and L5 spinal nerve transected mice (L5) received daily injections of 10 mg/kg propentofylline (PPF) or saline intraperitoneally, beginning one hour prior to surgery. Preventative treatment with propentofylline (L5, PPF) resulted in a significant decrease in mechanical allodynia to both 0.008 g (FIG. 12A) and 0.02 g (FIG. 12B) von Frey filaments compared with L5 spinal nerve transected, saline (L5, sal) controls (#P<0.001). Mechanical allodynia was reported as the average number of paw withdrawal out of 10+SEM (n=5 to 8 per group). Day 0 represents pre-injury responses. **P<0.01, ***P<0.001 versus sham, saline group; αP<0.05, +P<0.01 versus L5, saline group.

FIG. 13 depicts the effects of propentofylline on expression of glutamate transporters in spinal cord tissue from transgenic mice. Propentofylline reversed the glutamate transporter alterations induced by L5 spinal nerve transection. FIG. 13A shows quantitation of eGFP-GLT-1-positive puncta in the spinal cord dorsal horn on day 12 post-L5 transection, where it is seen that there was a significant decrease in the L5/saline treatment group (**P<0.01 versus sham/saline group) which was reversed with propentofylline administration (**P<0.01 versus L5/propentofylline treatment group). FIG. 13B shows that DsRed-GLAST expression trended towards a decrease in the L5/saline treatment group. Propentofylline significantly enhanced the number of DsRed-GLAST-positive puncta ipsilateral to the injury (*P<0.05 versus L5/saline treatment group). Results are expressed as the group mean±SEM.

### DETAILED DESCRIPTION OF THE INVENTION

It has now been found that propentofylline acts by two specific mechanisms to affect mediators of acute and chronic pain, e.g., neuropathic pain or inflammatory pain. These mechanisms involve differentiation of astrocytes to a is homeostatic, mature phenotype that is capable of glutamate clearance and alteration of glial glutamate transporters as a way to control aberrant glial activation. The present invention is therefore a method for design and identification of compounds that can be used to prevent or treat acute and chronic pain, e.g., neuropathic pain or inflammatory pain, in animals and humans.

Previous studies had shown that propentofylline, a methylxanthine derivative, exhibits anti-allodynic properties in an L5 spinal transaction model of neuropathic pain (Sweitzer, S. M. et al. 2001. J. Pharmacol. Exp. Ther. 297:1210-1217). Studies were performed to determine if propentofylline was capable of enhancing the expression of glutamate transporters in primary cultured astrocytes, cells that exist in a de-differentiated, activated state comparable to that seen after insult to the central or peripheral nervous system (Colburn, R. W. et al. 1999. Exp. Neurol. 157:289-304; Hashizume, H. et al. 2000. Spine 25:1206-1217). Experiments also were performed to determine if differentiation of astrocytes alone was sufficient to suppress LPS-induced release of chemokines which may play a role in neuropathic pain-related sensitization (White, F. A. et al. 2005. Nat. Rev. Drug Discov. 2:973-985).

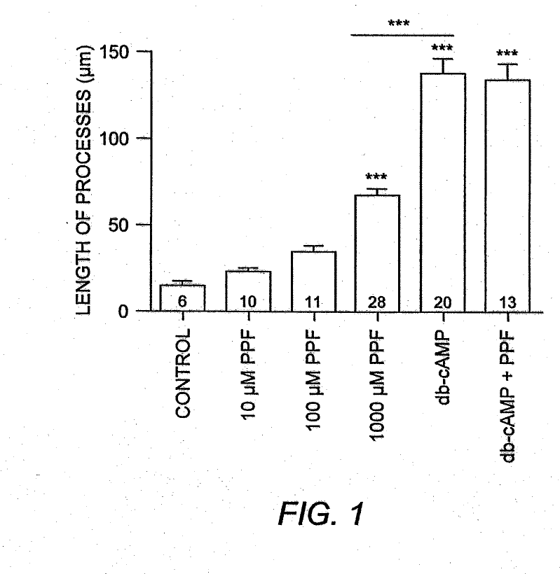

The effect of propentofylline on astrocyte morphology was investigated in primary cortical cultures of cells from cortices of 1 to 3 day old rats. After 3 days or 7 days of propentoftylline treatment, cells underwent a dose-dependent shift from a flat, polygonal shape to a stellate, process-bearing shape, as seen by phase contrast microscopy. Although both db-cAMP and high-dose propentofylline were able to differentiate cells, the resulting morphology differed. A characteristic propentofylline-differentiated astrocyte is highly ramified with thick, short processes, while db-cAMP treatment caused astrocytes to extend fewer, longer processes. Quantification of process length confirmed that db-cAMP treatment caused astrocytes to extend significantly longer processes than PPF (see FIG. 1; P<0.001 vs. 1000 μM propentofylline). Immunocytochemistry revealed this change in phenotype more clearly as one that resembled, but did not identically mimic, the identified phenotype, a phenotype that had been seen previously with db-cAMP treatment (Pollenz, R. S. and K. D. McCarthy. 1986. J. Neurochem. 47:9-17). Staining for GFAP demonstrated that cells had extended processes and furthermore, there was a concomitant increase in GLT-1 staining that co-localized with GFAP. It was noted that control cells had very little GLT-1 expressed, which was consistent with previous work (Schlag, B. D. et al. 1998. Mol. Pharmacol. 53:355-369) showing that cultured astrocytes express very low levels of this specific glutamate transporter. At day 7 of treatment, the GLT-1 is distributed in a punctate manner, in the cell body and along processes. Cells expressing the highest levels of GLT-1 also expressed lower levels of GFAP. This finding is consistent with the idea that quiescent (low GFAP expressing) astrocytes are competent for glutamate clearance from the synapse.

These results confirmed that control astrocytes are immunoreactive for GFAP and GLAST protein, at the same time showing that propentofylline treatment slightly increased GLAST staining, as did db-cAMP, but that the increased levels of GLT-1 were more pronounced.

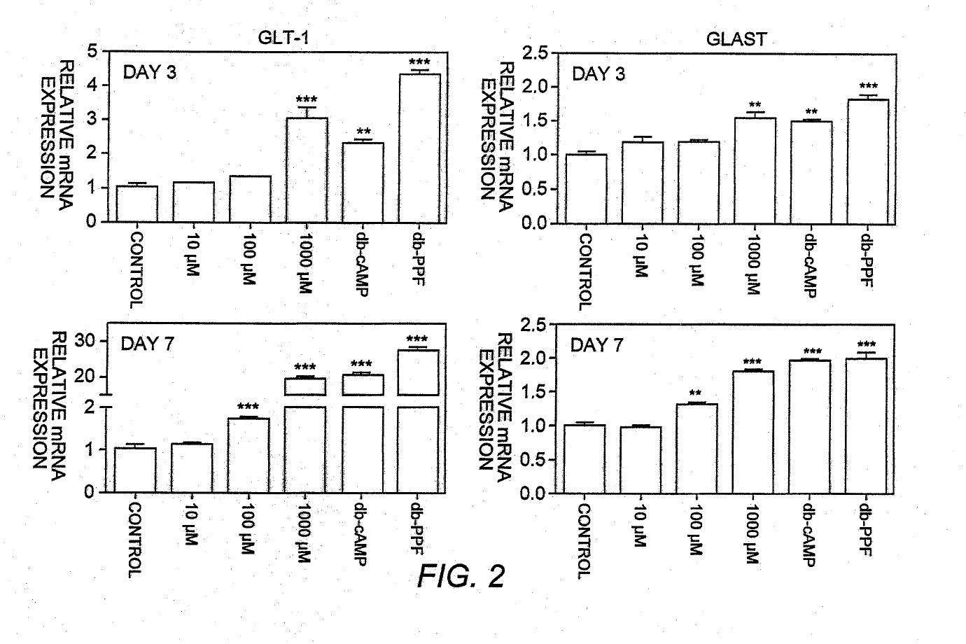

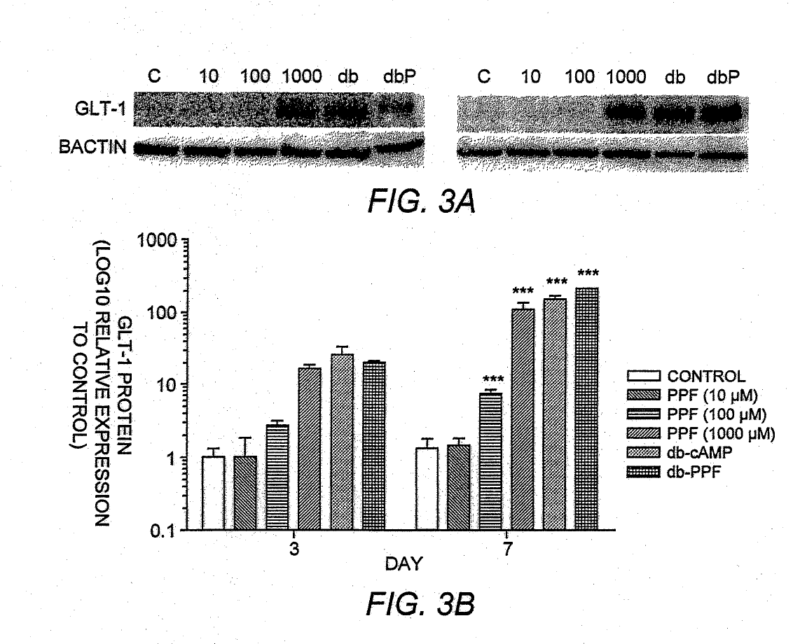

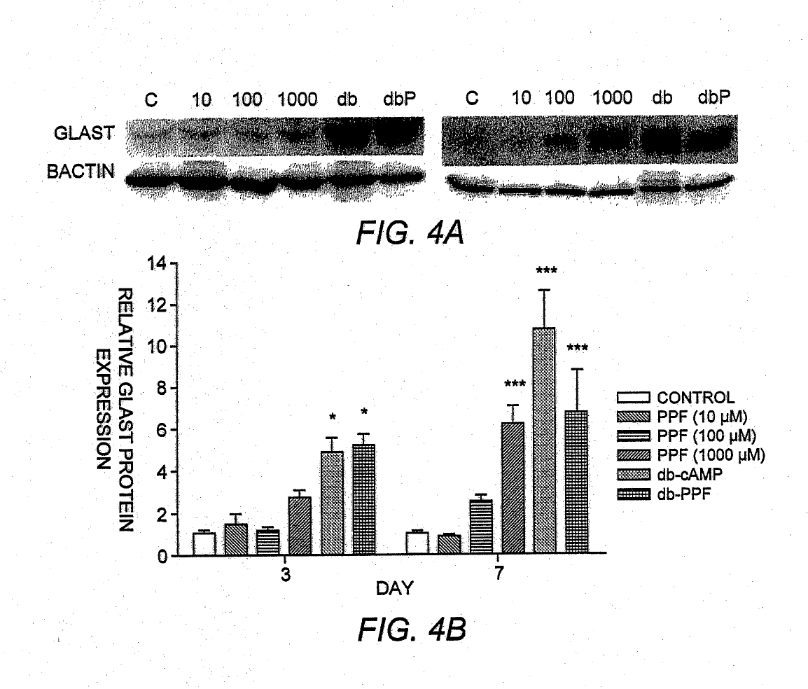

Alterations in glutamate transporter levels were then determined in the cultured, propentofylline-treated astrocytes using Real Time RT-PCR and western blot analysis. Propentofylline treatment dose-dependently induced GLT-1 and GLAST at the mRNA level (FIG. 2). By day 3, high dose propentofylline was associated with a 3-fold increase in GLT-1 mRNA while at day 7 a 20-fold induction was observed. At both days 3 and 7, propentofylline treatment resulted in a 1.5-2-fold increase in GLAST mRNA. Protein level changes mimicked that seen at the transcriptional level; after 3 days of treatment, GLT-! levels were increased by about 10-fold and by 7 days, high dose propentofylline treatment produced a 100-fold increase in GLT-1 levels, increases which were similar to that seen with db-cAMP treatment (FIG. 3). Corresponding to the immunocytochemical findings, results from western blot analyses demonstrated propentofylline-induced alterations in GLAST that were more modest; 7-day treatment with 1000 μM propentofylline induced a 6-fold increase in GLAST (FIG. 4). db-cAMP induced a greater increase in GLAST than high dose propentofylline, suggesting that the ultimate phenotype induced by db-cAMP and propentofylline differ in morphology and functional gliochemical characteristics.

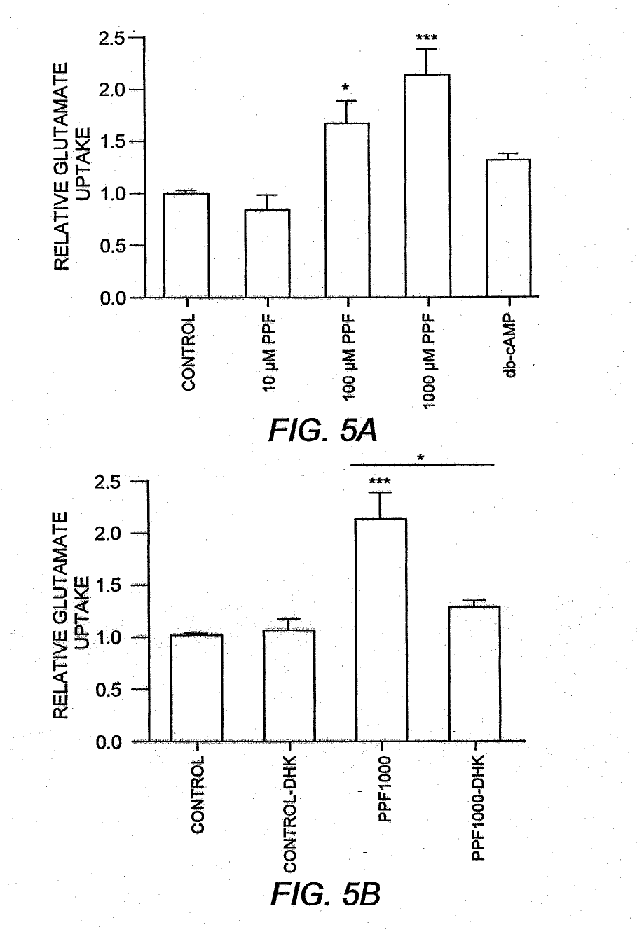

To determine whether the observed increase in glutamate transporters had a functional consequence, in vitro glutamate uptake studies were performed. Treatment of cells with 100 μM propentofylline for 7 days led to a 1.7-fold increase in glutamate uptake (FIG. 5), while 1000 μM propentofylline treatment resulted in a 2.1-fold increase (FIG. 5) in Na⁺-dependent glutamate transport, indicating the effects were dose-dependent. Previously it had been shown that db-cAMP induced high levels of GLT-1 protein expression and led to a 2-fold increase in glutamate transport activity (Schlag, B. D. et al. 1998. Mol. Pharmacol. 53:355-369), however, this uptake was found to be DHK-insensitive. Therefore, experiments were performed to determine if the increased uptake observed with propentofylline treatment was GLT-1-mediated. In contrast to db-cAMP, the increased expression of GLT-1 protein was found to have a functional consequence, as the [³H]-glutamate transport was sensitive to DHK inhibition (FIG. 5B).

In order to ensure that the morphological effects of propentofylline on astrocytes were due to specific pharmacological effects and not due to toxicity, experiments were performed to determine if the phenotype change observed was reversible. Cultured astrocytes treated for 7 days with high dose propentofylline or db-cAMP demonstrated the expected morphological change. However, a further 7 day washout period (during which astrocytes received control media only) reinstated the “activated” state of astrocytes (polygonal, flat, non-process bearing cells). The reversal was more complete in propentofylline-treated cells compared with db-cAMP treated cells, highlighting the fact that propentofylline is a glial modulating agent whose effects are reversible.

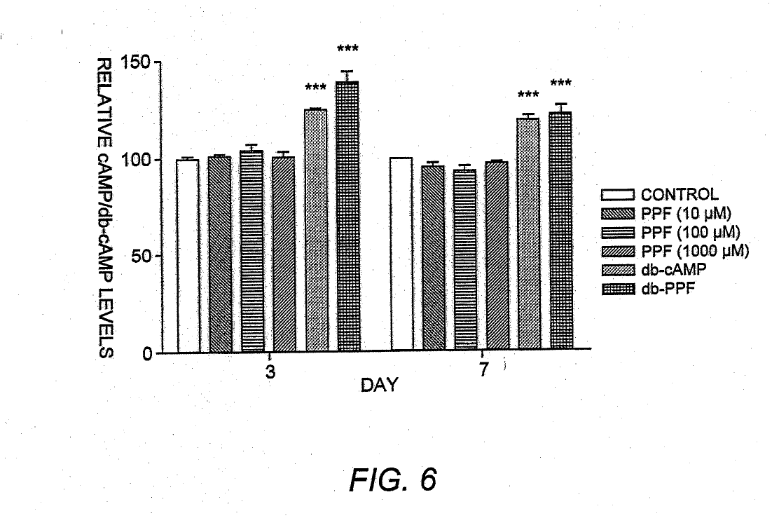

Finally, since one postulated mechanism of action of propentofylline is inhibition of LAMP phosphodiesterase, the effect of propentofylline on cAMP levels in treated astrocytes was examined. At days 3 and 7, db-cAMP treatment led to an increase in competition for PKA binding attributable to the db-cAMP itself, manifesting as an increase in relative cAMP/db-cAMP levels (FIG. 6). In contrast, even high doses of propentofylline did not enhance cAMP activity indicating that propentofylline may have caused a transient increase in cAMP that was no longer detected at days 3 and 7.

A hallmark of glial cell activation is the release of proinflammatory cytokines, chemokines and other neuronal sensitizing factors. Therefore, additional experiments were performed to determine if astrocyte differentiation alone is sufficient to inhibit cytokine and chemokine release from astrocytes stimulated with LPS.

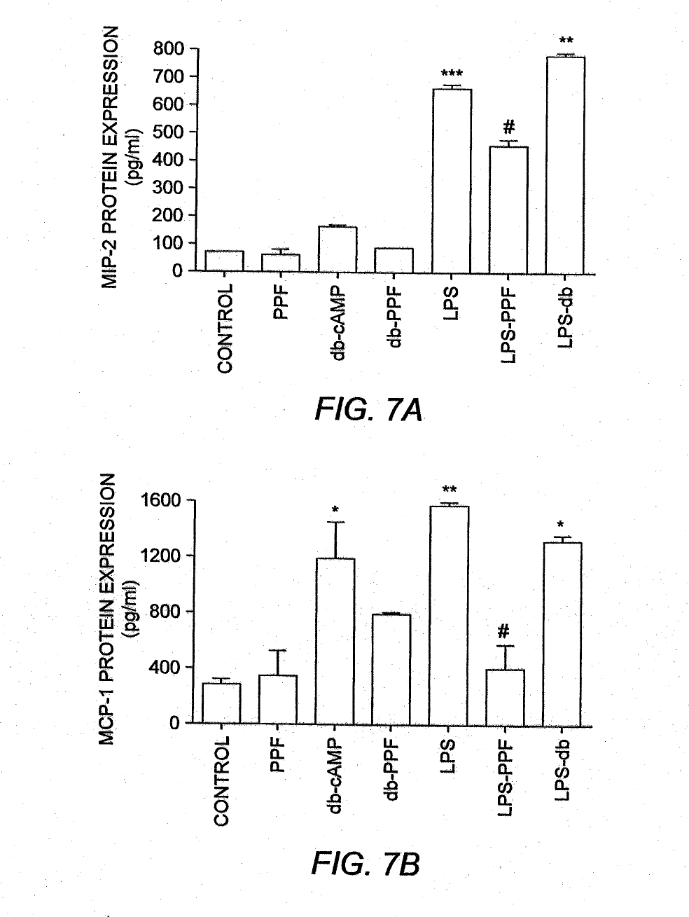

Seven-day treatment with LPS clearly induced MIP-2 (CXCL2) (FIG. 7A) and MCP-1 (CCL2) (FIG. 7B) release from cultured astrocytes. Concomitant treatment with 250 μM db-cAMP, a dose previously shown to differentiate astrocytes, had no effect on LPS-induced chemokine release. In contrast, high dose propentofylline treatment significantly attenuated both MIP-2 (CXCL2) and MCP-1 (CCL2) release from astrocytes (FIG. 7). db-cAMP treatment alone significantly increased MCP-1 (CCL2) above basal levels, while propentofylline treatment by itself in cultured astrocytes had no effect, again indicating that the differentiated phenotype obtained is functionally distinct.

The results demonstrated that there is a link between aberrant astrocytic activation and dysregulation of the glutamate uptake system. Further, the results showed that propentofylline, a methylxanthine derivative that exhibits anti-allodynic properties in a neuropathic pain model, induces an astrocyte phenotype switch from activated to differentiated and homeostatic. It has also been shown that propentofylline concomitantly induced transcription and translation of the glutamate transporters, GLT-1 and GLAST. These data provide a mechanism for exploitation in the development of compounds to treat pain.

In order to examine the role of glial cell glutamate transporters in behavioral sensitivity following nerve injury, experiments were performed in the L5 spinal nerve transection model of neuropathic pain, a model that has been demonstrated to reliably produce mechanical allodynia (behavioral hypersensitivity; Sweitzer, S. M. et al. 2001. J. Pharmacol. Exp. Ther. 297:1210-1217). Experiments were directed to examination of changes at both the transcriptional and translational levels.

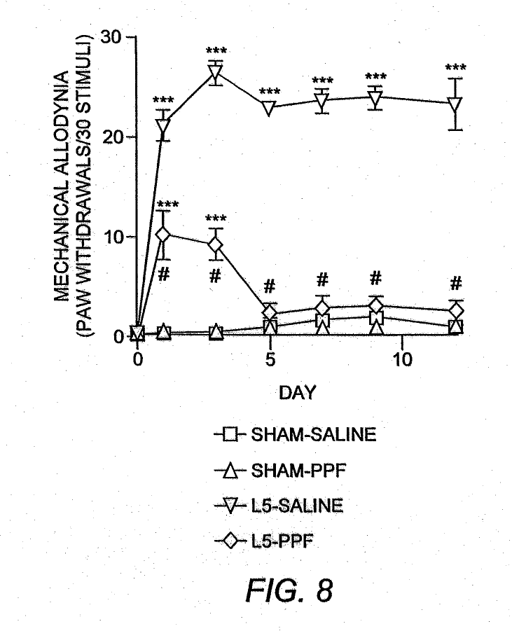

The L5 spinal nerve transection model of mononeuropathy has been previously shown to induce robust mechanical allodynia in the ipsilateral hindpaw (Colburn, R. W. et al. 1999. J. Neuroimmunol. 79:163-175). Therefore, rats were surgically prepared as described in the art. Rats receiving L5 spinal nerve transection displayed significantly greater paw withdrawals to a 12 g Von Frey filament starting at day 1 post-transection as compared with normal animals (FIG. 8). Daily treatment with propentofylline initiated one hour prior to transection robustly inhibited the development of mechanical allodynia at all time points tested (FIG. 8). In addition, from day 5 onward, the L5 spinal nerve transection group receiving propentofylline was no longer discernable from the normal group (P>0.05, normal vs. L5, propentofylline for day 5, 7, 9 and 12). These results further demonstrate an anti-allodynic effect upon propentofylline administration.

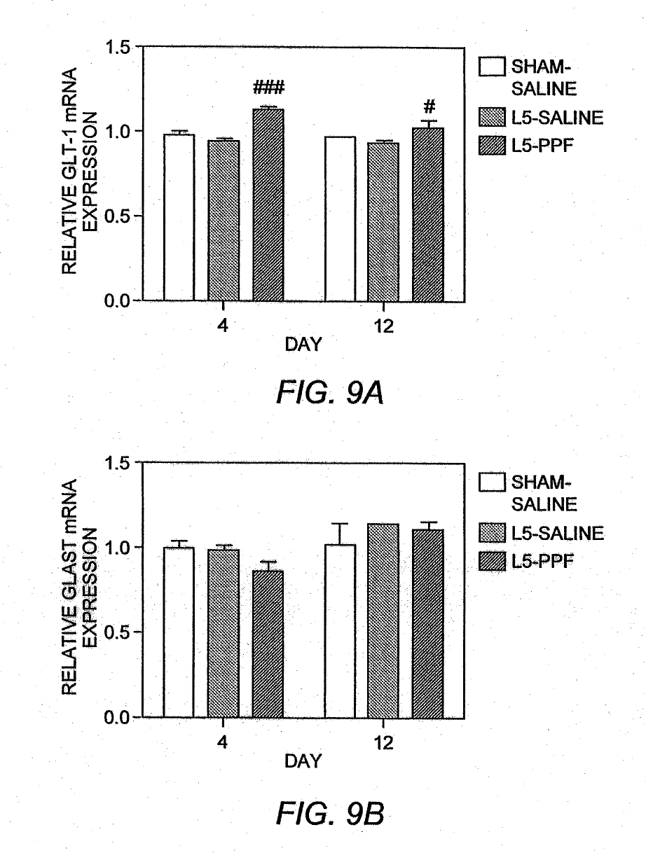

Experiments were then performed to examine the effects of propentofylline treatment on GLT-1 mRNA levels in cells of animals surgically transected, with lumbar spinal cord tissue collected on post-transection days 4 or 12. RT-PCR analysis of the tissue samples was carried out in order to determine if propentofylline modulated glutamate transporter expression at the transcriptional level. No difference was observed in GLT-1 mRNA expression between sham and L5 spinal nerve transected groups at either time point (FIG. 9A, Sham vs. L5). However, rats receiving daily intrathecal propentofylline expressed significantly increased levels of GLT-1 mRNA at day 4 (**P<0.01 vs. sham; ^{#}P<0.001 vs. L5) and day 12 (*P<0.05 vs. L5). GLAST mRNA levels in L5 spinal nerve transected rats were unchanged compared with sham. Propentofylline treatment led to a slight decrease in GLAST mRNA at day 4 compared to L5 spinal nerve transection alone (FIG. 9B).

Studies have shown that astrocytic activation, as evidenced by an increase in GFAP staining, is enhanced in the dorsal horn of the spinal cord after L5 spinal nerve transection (Tawfik, V. L. et al. 2005. J. Pharmacol. Exp. Ther. 313:1-9). Experiments were performed that confirmed this finding and additionally showed enhanced activation of astrocytes at 12 days post-L5 spinal nerve transection with more intense GFAP immunoreactivity in the ipsilateral dorsal horn.

Analysis was subsequently conducted to assess levels of the glutamate transporters, GLT-1 and GLAST, in lumbar spinal tissue to determine whether propentofylline was capable of altering glutamate transporter protein levels. In the sham surgery group, staining for GLT-1 was observed diffusely in the gray matter of the spinal cord, with higher levels in laminae I and II. Twelve days after L5 spinal nerve transection, GLT-1 immunoreactivity was decreased on the side ipsilateral to the lesion. In contrast, propentofylline treated rats demonstrated no qualitative difference in GLT-1 between ipsilateral and contralateral dorsal horns. Therefore, the L5 spinal nerve injury-induced decrease in GLT-1 was abolished by propentofylline treatment. GLAST immunoreactivity in sham surgery rats was almost exclusively localized to the upper dorsal horn laminae. After L5 spinal nerve transection, decreased GLAST was observed in the ipsilateral dorsal horn. Propentofylline treatment did not enhance GLAST immunoreactivity on the ipsilateral side.

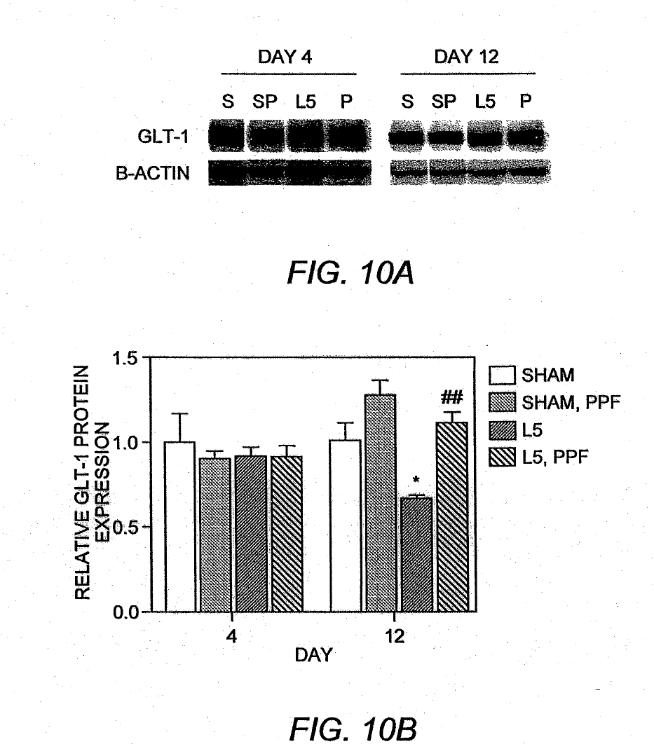

To quantitatively determine the effects of propentofylline on glutamate transporter protein expression, western blot analysis of ipsilateral lumbar spinal cord was conducted. There was no change in GLT-1 protein observed in the L5 spinal nerve transection group at day 4 (FIG. 10). There was a decrease in GLT-1 protein at day 12 post-transection compared to sham, although this did not reach statistical significance. Propentofylline-treated rats exhibited similar levels of GLT-1 protein as compared to the sham control group at both time points. On day 12, however, GLT-1 protein was significantly elevated in injured rats receiving propentofylline compared to those receiving L5 spinal nerve transection alone (FIG. 10; *P<0.05 vs. L5), indicating a role for GLT-1 in propentofylline-induced anti-allodynia.

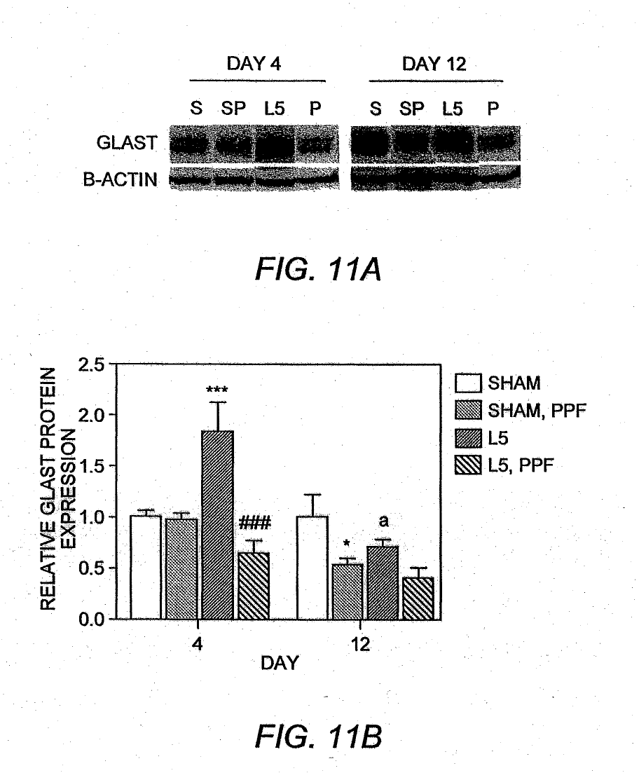

GLAST protein levels exhibited a different pattern following injury and drug treatment. Four days after L5 spinal nerve transection, GLAST protein levels were significantly elevated above levels seen in sham animals (FIG. 11, **P<0.001 vs. sham), and by day 12, GLAST density trended towards being lower than in sham controls. There was a significant decrease in GLAST in the propentofylline group as compared with the saline-treated, transected rats at day 4 (^{#}P<0.001 vs. L5) and a slight decrease at day 12 (not statistically significant). These data demonstrated differential modulation of GLT-1 and GLAST by propentofylline in a rodent pain model.

In addition to studies using a rat model of pain for examining the role of glutamate transporters in the therapeutic response (analgesic) of propentofylline treatment, studies were also performed in a double transgenic reporter mouse line that expressed eGFP-GLT-1/DsRed-GLAST. Use of the transgenic mouse model further defined the effect of propentofylline on glutamate transporters, GLT-1 and GLAST. The animals were injected intraperitoneally with 10 mg/kg propentofylline in sterile saline or saline vehicle alone (n=5 to 8 per group). The first injections were administered one hour prior to L5 spinal nerve transection and continued daily in the evening (between 5 and 7 PM) until day 12 post-transection. Mice received either an L5 spinal nerve transection or sham surgery on day 0. The development of mechanical allodynia was monitored on days 1, 3, 5, 7, 9 and 12 as described for rats previously. The monitoring was performed in the morning at approximately 15 hours post-propentofylline or saline injection. Mice were transcardially perfused on day 12 post-transection, spinal cords were removed, and the tissue was processed for immunohistochemistry.

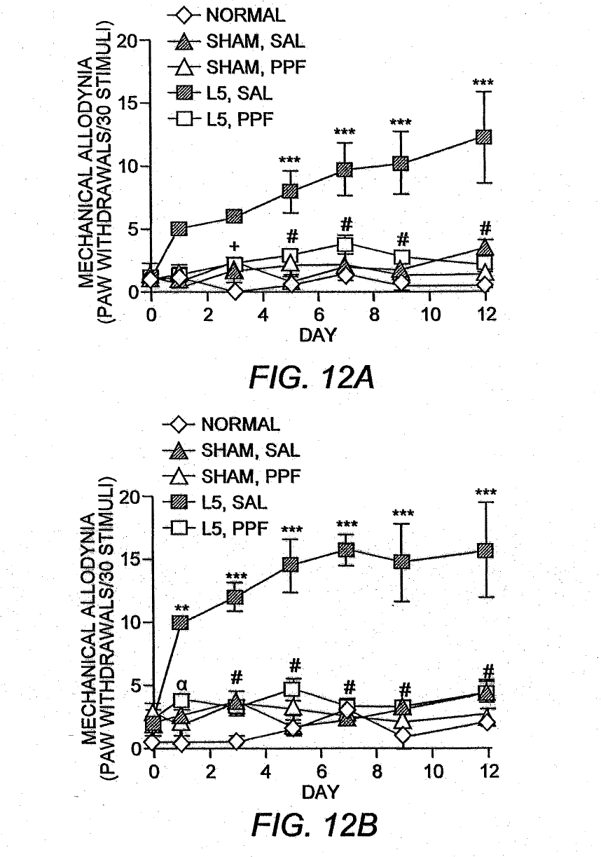

As was shown previously in rats, daily treatment with propentofylline, initiated one hour before transection, robustly inhibited the development of mechanical allodynia in mice (FIG. 12). After L5 spinal nerve transection, mice developed mechanical allodynia to both 0.008 g (FIG. 12A: ***P<0.001; Sham, saline group vs. L5, saline group) and 0.02 g (FIG. 12B: **P<0.01, ***P<0.001; Shan, saline group vs. L5. saline group) von Frey filaments. The effects of propentofylline to inhibit mechanical allodynia were evident in mice starting at day 1 for the 0.02 g filament (αP<0.01, #P<0.001; L5, saline group vs. L5, propentofylline group) and day 3 for the 0.008 g filament (+P<0.05, #P<0.001; L5, saline group vs. L5, propentofylline group). Further, results showed that the L5, propentofylline-treated mice remained similar to the sham control mice at every time point for both filaments.

With these in vivo data collected, further experiments were performed in the transgenic mice to more clearly define the effects of propentofylline treatment on GLT-1 and GLAST in the spinal cord. As discussed above, following in vivo testing, mice were transcardially perfused and tissue was fixed with paraformaldehyde. Lumbar spinal cord sections were identified and post-fixed in paraformaldehyde. After one week in sucrose to prevent freeze fracture, spinal cord segments were frozen in dry ice, mounted and prepared with cryostat sectioning. Sections were then subjected to immunohistochemical analysis. The transgenic mouse line used exhibits a distinct pattern of GLT-1 and GLAST expression in the spinal cord that is characterized by both punctate, perinuclear expression as well as diffuse, cytoplasmic staining. In order to quantitate the relative number of eGFP (marker for GLT-1) or DsRed (marker for GLAST) puncta in each spinal cord dorsal horn, images using a fluorescence microscope were taken with a Q-Fire cooled camera (397 ms exposure). Images taken with the 488 nm laser, showing eGVP, were altered by decreasing the brightness to −35 units and increasing the contrast to +71 units, which resulted in images with bright, eGFP positive puncta that were easily identifiable. Images taken using the 555 nm laser, showing DsRed staining, were altered by decreasing the brightness to −12 units and increasing the contrast to +65 units, which resulted in images with easily identifiable DsRed puncta. An observer blind to the treatment groups was used to count the relative number of GLT-1 or GLAST positive puncta in the ipsilateral (injured) dorsal horn versus the contralateral (non-injured) dorsal horn. Results showed that the transgenic mice showed two types of staining in the dorsal horn of the spinal cord. For both reporters (GLT-1, green color; GLAST, red color), diffuse, cytoplasmic and punctate, perinuclear expression was observed that overlapped with GFAP (blue color) in some, but not all cases. As was seen previously in rats, L5 spinal nerve transection led to an enhancement of GFAP, most notably on the side ipsilateral to the injury. Additionally, the results showed that propentofylline treatment suppressed injury-induced GFAP expression in the dorsal horn upper laminae of the mice.

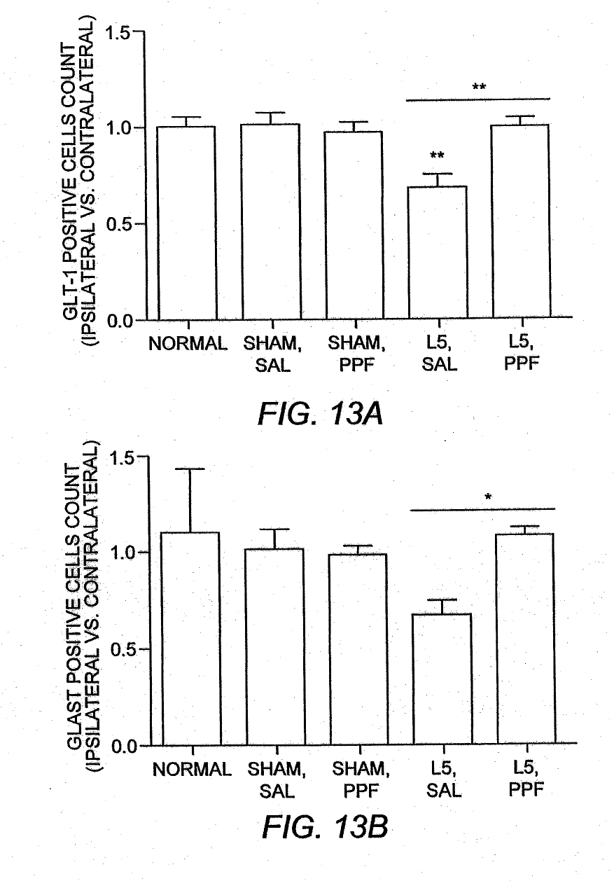

Each mouse exhibited a unique pattern of eGFP-GLT-1 expression in the dorsal horn of the spinal cord. While there was significant animal-to-animal variation in the number of eGFP-positive puncta in each mouse; there was consistency across the dorsal horns examined for a given animal, making it possible to compare injured versus non-injured side effects. Both the sham/saline group and the sham/propentofylline groups displayed equivalent numbers of eGFP-GLT-1-positive puncta in both spinal cord dorsal horns, indicating that propentofylline treatment did not lead to further increases in GLT-1 above normal expression levels. In contrast, L5 spinal nerve transaction was associated with a decrease in the number of eGFP-GLT-1-positive puncta in the ipsilateral dorsal horn. Treatment of injured mice (L5/propentofylline group) with propentofylline restored levels of eGFP-GLT-1-positive puncta to that of the contralateral side. Quantitation of immunofluorescent puncta (FIG. 13) demonstrated that L5 spinal nerve transection led to a significant decrease in eGFP-GLT-1-positive puncta compared to the sham/saline treated group (FIG. 13A, **P<0.01). Daily propentofylline treatment increased the number of eGFP-GLT-1-positive puncta on the injured side and restored the ipsilateral versus the contralateral ratio to 1 (**P<0.01; L5, saline group versus L5, propentofylline group).

It has previously been shown that the GLAST transporter is expressed on a separate population of cells I the spinal cord and is expressed at a much lower level than GLT-1 (Regan et al. 2007. J. Neuroscience, in press). The data provided herein are consistent with that finding, where very few DsRed-GLAST puncta were observed in the lumbar dorsal horns and cytoplasmic-type expression of the transgene was considerably lower than that of eGFP-GLT-1. As was observed with GLT-1 expression, GLAST expression was unaffected in sham/saline mice or in sham/propentofylline mice. Although the absolute number of DsRed-GLAST puncta varied significantly between animals, the ipsilateral versus contralateral ratio remained close to 1 for any individual mouse. As was seen with GLT-1, L5 spinal nerve transection led to a decrease in DsRed-GLAST puncta on the ipsilateral side and propentofylline treatment increased the levels back to that seen in the contralateral side. When quantitated, a significant effect of propentofylline on the ipsilateral versus contralateral ratio of GLAST puncta was observed (FIG. 13B, *P<0.050 L5, saline group versus L5, propentofylline group).

The data presented herein demonstrate a novel mechanism for compounds that can be used to treat acute and chronic pain, e.g., neuropathic pain or inflammatory pain. Propentofylline, a methylxanthine derivative, is shown to be capable of transcriptional and translational regulation of glutamate transporters, GLT-1 and GLAST, and also capable of inducing a functionally significant enhancement of glutamate transport in astrocytes. In this way propentofylline has been shown to act as an anti-allodynic agent by targeting injury-induced, aberrantly activated astrocytes and restoring the cells to their mature, differentiated GLT-1-expression state. This effect leads to attenuation of neuronal glutamate receptor activation and ectopic firing that is related to central sensitization.

Accordingly, the present invention relates to methods for identifying compounds that can be used to prevent or treat acute and chronic pain as well as methods for preventing, treating, inhibiting, and/or alleviating such pain that involves administration of a compound that enhances astrocytic glutamate transporter GLT-1 expression or activity. As used in the context of the present invention, acute and chronic pain, e.g., neuropathic or inflammatory pain, is pain that originates from a damaged nerve or nervous system.

Generally, an agent of the present invention is administered to an animal or human identified as being in need of such prevention or treatment using routes (e.g., injection, infusion, or inhalation) and dosages that are determined to be appropriate by those of skill in this art. As used in the context of the present invention, an individual in need of treatment can include a human, zoo animal, companion animal, laboratory animal or livestock. The methods of the present invention comprise administering the agents and/or pharmaceutical compositions by a variety of routes that would include but not be limited to orally, sublingually, subcutaneously, intramuscularly, intravenously, intranasally, by inhalation, or transmucosally.

For parenteral applications, particularly suitable are oily or aqueous solutions, as well as suspensions, emulsions, or implants, including suppositories.

For oral application, particularly suitable are tablets, troches, liquids, drops, capsules, caplets and gel caps.

The compositions intended for oral use may be prepared according to any method known in the art and such compositions may contain one or more agents selected from the group consisting of inert, non-toxic pharmaceutical excipients which are suitable for the manufacture of tablets. Such excipients include, for example, an inert diluent such as lactose, granulating and disintegrating agents such as cornstarch, binding agents such as starch, and lubricating agents such as magnesium stearate. The tablets may be uncoated or they may be coated by known techniques, in some instances in order to delay release of the active ingredients. Formulations for oral use may also be presented as hard gelatin capsules wherein the active ingredient is mixed with an inert diluent. Aqueous suspensions are contemplated to contain the drug and one or more excipients suitable as suspending agents, such as, for example, pharmaceutically acceptable synthetic gums (e.g., hydroxypropylmethylcellulose or natural gums). Oily suspensions may be formulated by suspending the aforementioned combinations of drugs in a vegetable oil or mineral oil. The oily suspensions may contain a thickening agent such as beeswax or cetyl alcohol. Syrup, elixir, or the like can be used wherein a sweetened vehicle is employed.

Injectable suspensions may also be prepared, in which case appropriate liquid carriers, suspending agents and the like may be employed. It is also possible to freeze-dry the active compounds and use the obtained lyophilized compounds, for example, for the preparation of products for injection.

An effective amount of agent administered is defined as an amount which prevents, attenuates, or reduces behavioral hypersensitivity associated with acute and chronic pain, e.g., neuropathic pain or inflammatory pain. Behavioral hypersensitivity of pain may include sensations that are sharp, aching, throbbing, gnawing, deep, squeezing, or colicky in nature and can be measured by, for example, exposure to thermal hyperalgesia or mechanical hyperalgesia.

As will be understood by those of skill in this art, the specific dose level for any particular patient will depend on a variety of factors, including the activity of the specific compound employed; the age, body weight, general health, and sex of the individual being treated; the time and route of administration; the rate of excretion; other drugs that have previously been administered; and the severity of the particular disease undergoing therapy.

In certain embodiments, the dosage of the agents will generally be in the range of about 0.01 ng to about 10 g per kg body weight, specifically in the range of about 1 ng to about 0.1 g per kg, and more specifically in the range of about 100 ng to about 10 mg per kg. In certain embodiments, dosage amounts may also be in terms of mg/m² of a person. In certain embodiments, the dosage of the agents will generally be in the range of about 10 to 100 mg/m², 20 to 90 mg/m², 30 to 80 mg/m², 40 to 70 mg/m², or 50 to 60 mg/m². In another embodiment the dosage of the agents will generally be about 60 mg/m².

An effective dose or amount, and any possible affects on the timing of administration of the formulation, may need to be identified for any particular composition of the present invention. This may be accomplished by routine experiment, using one or more groups of animals (preferably at least 5 animals per group), or in human trials if appropriate. The effectiveness of any agent and method of treatment or prevention may be assessed by administering the agent and assessing the effect of the administration by measuring one or more applicable indices, and comparing the post-treatment values of these indices to the values of the same indices prior to treatment.

The precise time of administration and amount of any particular agent that will yield the most effective treatment in a given patient will depend upon the activity, pharmacokinetics, and bioavailability of an agent, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including agent, amounts, times of administration and formulation, may be optimized according to the results of such monitoring. The patient may be periodically reevaluated to determine the extent of improvement by measuring the same parameters. Adjustments to the amount(s) of agent administered and possibly to the time of administration may be made based on these reevaluations.

Treatment may be initiated with smaller dosages which are less than the optimum dose of the agent. Thereafter, the dosage may be increased by small increments until the optimum therapeutic effect is attained.

Toxicity and therapeutic efficacy of agents may be determined by standard pharmaceutical procedures in cell cultures or experimental animals, e.g., for determining the LD₅₀ and the ED₅₀.

The data obtained from cell culture assays and animal studies may be used in formulating a range of dosage for use in humans. The dosage of any agent lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays.

The agents of the present invention may be in a single dosage form or in a multiple dosage form. When the agents of the present invention are in multiple dosage forms, the dosage forms may be administered to a subject concurrently or sequentially. When the dosage forms are administered sequentially, conceivably any time period may apply between dosages. Generally the time period applied will be in accordance with a physician's directions. In one embodiment, the time period between dosages may be 30 seconds, 1 minute, 5 minutes, 1 hour, 2 hours, or more.

The method of the present invention is particularly useful for preventing and/or treating pain associated with conditions that would include but not be limited to neuropathies, polyneuropathies (e.g., as in diabetes and trauma), neuralgias (e.g., post-zosterian neuralgia, postherpetic neuralgia, trigeminal neuralgia, algodystrophy, and HIV-related pain); musculo-skeletal pain such as osteo-traumatic pain, arthritis, osteoarthritis, spondylarthritis as well as phantom limb pain, back pain, vertebral pain, post-surgery pain; cancer-related pain; vascular pain such as pain resulting from Raynaud's syndrome, Horton's disease, arteritis, and varicose ulcers; as well as pain associated with multiple sclerosis, Crohn's Disease, and endometriosis.

It is contemplated that the agents of the present invention can be used alone or in combination with other treatments known to alleviate pain. In one embodiment, the other treatment is administration of an agent to treat pain, such as an analgesic. In a further embodiment, the present invention relates to a composition comprising propentofylline and a formulation designed to deliver the propentofylline to the stomach, duodenum, small intestine, colon, rectum, vagina, nasal passageway, uterus, ovaries, or Fallopian tubes.

In another aspect, the present invention relates to a method of treating acute and chronic pain by administering a composition comprising propentofylline and/or another therapeutic agent. In a further embodiment, the therapeutic agent is an analgesic. In another embodiment, the analgesic is selected from the group consisting of chlorobutanol, clove, eugenol, alfentanil, allylprodine, alphaprodine, anileridine, benzylmorphine, bezitramide, buprenorphine, butorphanol, clonitazene, codeine, codeine methyl bromide, codeine phosphate, codeine sulfate, desomorphine, dextromoramide, dezocine, diampromide, dihydrocodeine, dihydrocodeinone enol acetate, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone hydrochloride, metopon, morphine, morphine hydrochloride, morphine sulfate, myrophine, nalbuphine, narceine, nicomorphine, norlevorphanol, normethadone, normorphine, norpipanone, opium, oxycodone, oxymorphone, papaveretum, pentazocine, phenadoxone, phenazocine, phenoperidine, piminodine, piritramide, proheptazine, promedol, propiram, propoxyphene, remifentanil, sufentanil, tilidine, aceclofenac, acetaminophen, acetaminosalol, acetanilide, acetylsalicylsalicylic acid, alclofenac, alminoprofen, aloxiprin, aluminum bis(acetylsalicylate), aminochlorthenoxazin, 2-amino-4-picoline, aminopropylon, aminopyrine, ammonium salicylate, amtolmetin guacil, antipyrine, antipyrine salicylate, antrafenine, apazone, aspirin, benorylate, benoxaprofen, benzpiperylon, benzydamine, bermoprofen, bromfenac, p-bromoacetanilide, 5-bromosalicylic acid acetate, bucetin, bufexamac, bumadizon, butacetin, calcium acetylsalicylate, carbamazepine, carbiphene, carsalam, chlorthenoxazin(e), choline salicylate, cinchophen, ciramadol, clomctacin, clonixin, cropropamide, crotcthanudc, dexoxadrol, dilcnanuzole, ditlunisal, dihydroxyaluminum acetylsalicylate, dipyrocetyl, dipyrorre, fanurfazone, entcnanuc acid, epirizole, etersalatc, elhcnzamide, ethoxazcne, etodolac, felbinac, fenoprofen, floctafenine, flufenamic acid, fluoresone, flupirtine, fluproyuazone, flurbiprofen, fosfosal, gentisic acid, glafenine, ibufenac, imidazole salicylate, indomethacin, indoprofen, isofezolac, isoladol, isonixin, ketoprofen, ketorolac, p-lactophenetide, lefetamine, lornoxicam, loxoprofen, lysine acetylsalicylate, magnesium cetylsalicylate, methotrimeprazine, metofoline, mofezolac, morazone, morpholine salicylate, naproxen, nefopam, nifenazone, 5′-nitro-2′-propoxyacetanilide, parsalmide, perisoxal, phenacetin, phenazopyridine hydrochloride, phenocoll, phenopyrazone, phenyl acetylsalicylate, phenyl salicylate, phenyramidol, pipebuzone, piperylone, propacetamol, propyphenazone, ramifenazone, rimazolium metilsulfate, salacetamide, salicin, salicylamide, salicylamide O-acetic acid, salicylsulfuric acid, salsalate, salverine, simetride, sodium salicylate, suprofen, talniflumate, tenoxicam, terofenamate, tetrandrine, tinoridine, tolfenamic acid, tramadol, tropesin, viminol, xenbucin, zomepirac, pregabalin, gabapentin, carbamazepine, NGX-4010, NP-1, amitriptyline, nortriptyline, ruboxistaurin, duloxetine, memantine, lamotrigine, REN-1654, Neurodex, Prosaptide, harkoseride, Liprostin, pirfenidone, AS-3201, TAK-428, QR-333, capsaicin, amitriptyline, amoxapine, chlomipramine, desipramine, doxepin, imipramine, nortriptyline, protriptyline, trimipramine, and combinations thereof.

In a further embodiment, the agents of the present invention further comprise a pharmaceutically acceptable carrier.

Agents that enhance or increase the expression or activity of astrocyte glutamate transporter GLT-1 can be identified in cell-free or cell-based screening assays. In one embodiment, such an assay involves the steps of contacting GLT-1 or a cell expressing GLT-1 with a test agent and measuring the expression or activity of GLT-1 in the presence and absence of the test agent, wherein an increase in the measured activity (e.g., glutamate transport) or expression of GLT-1 in the presence of the test agent, as compared to the measured activity or expression of GLT-1 in the absence of the test agent, indicates that the agent enhances GLT-1 expression or activity and is useful for preventing or treating acute and chronic pain.

The assay method of the invention can further be used to identify agents capable of inhibiting pain perception in an animal model system. Such an assay can involve the steps of administering a compound to be tested for its ability to prevent or treat pain intrathecally to a first rat that has been surgically transected at L5; anesthetizing the first rat and removing lumbar spinal cord tissue; measuring the level of GLT-1 in the lumbar spinal cord tissue from the first rat; administering a vehicle without the compound to be tested intrathecally to a second rat that has been surgically transected at L5; anesthetizing the second rat and removing lumbar spinal cord tissue; measuring the level of GLT-1 in the lumbar spinal cord tissue from the second rat; and comparing the levels of GLT-1 from the first and second rats, wherein an increase in the level of GLT-1 in the sample from the first rat as compared to the second rat is indicative of a compound that can be used to prevent or treat acute and chronic pain, e.g., neuropathic pain or inflammatory pain.

Screening assays of the invention can also be utilized to identify or characterize an agent which increases the expression of GLT-1. Thus, another embodiment embraces a method for identifying or characterizing an agent for regulating production of GLT-1, a method that involves contacting a first cell expressing GLT-1 with a test agent and measuring the GLT-1 mRNA or protein levels in the first cell as compared to a second cell expressing GLT-1 which has not been contacted with the test agent, wherein a higher measured level of GLT-1 mRNA or protein in the first cell compared to the second cell indicates that the test agent is useful for preventing or treating acute and chronic pain, e.g., neuropathic pain or inflammatory pain.

Such gene production or expression can be measured by detection of the corresponding RNA or protein, or via the use of a suitable reporter construct comprising a transcriptional regulatory element(s) of GLT-1, e.g., the GLT-1 promoter or the EAAT2 promoter fragment, operably-linked to a reporter gene. A first nucleic acid sequence is operably-linked with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since, for example, enhancers generally function when separated from the promoters by several kilobases and intronic sequences can be of variable lengths, some polynucleotide elements can be operably-linked but not contiguous. Transcriptional regulatory element is a generic term that refers to DNA sequences, such as initiation and termination signals, enhancers, and promoters, splicing signals, polyadenylation signals which induce or control transcription of protein coding sequences with which they are operably-linked. The expression of such a reporter gene can be measured on the transcriptional or translational level, e.g., by the amount of RNA or protein produced. RNA can be detected by, for example, northern analysis or by the reverse transcriptase-polymerase chain reaction (RT-PCR) method (see, for example, Sambrook, et al. 1989. Molecular Cloning: A Laboratory Manual, second edition, Cold Spring Harbor Laboratory Press, Cold Spring Harbor, N.Y., USA). Protein levels can be detected either directly using affinity reagents (e.g., an antibody or fragment thereof using methods such as described in Harlow and Lane. 1988. Antibodies: A Laboratory Manual, Cold Spring Harbor Laboratory Press, Cold Spring Harbor, N.Y. or a ligand which binds the protein) or by other properties (e.g., fluorescence in the case of green fluorescent protein) or by measurement of the protein's activity, which can entail enzymatic activity to produce a detectable product (e.g., with altered spectroscopic properties) or a detectable phenotype (e.g., alterations in cell growth). Suitable reporter genes include, but are not limited to, chloramphenicol acetyltransferase, beta-D galactosidase, luciferase, or green fluorescent protein. It is contemplated that microarray technology can be used to carry out this assay of the invention.

The assays can be employed either with a single test agent or a plurality of test agents or library (e.g., a combinatorial library) of test agents. In the latter case, synergistic effects provided by combinations of agents can also be identified and characterized. The above-mentioned agents can be used for increasing the expression or activity of GLT-1, for the prevention or treatment of acute and chronic pain, or as lead compounds for the development and testing of additional compounds having improved specificity, efficacy or pharmacological (e.g., pharmacokinetic) properties. In certain embodiments, one or a plurality of the steps of the screening/testing methods of the invention can be automated.

Agents which may be screened using the screening assays provided herein encompass numerous chemical classes, though typically they are organic molecules, preferably small organic compounds having a molecular weight of more than 100 and less than about 2,500 daltons. Agents comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents may also be found among biomolecules including peptides, agonistic antibodies, saccharides, fatty acids, steroids, purines, pyrimidines, derivatives, structural analogs or combinations thereof. For example cAMP analogs such as 8-Br-cAMP and db-cAMP could be screened in accordance with the instant assays and used in the prevention and treatment of acute and chronic pain, e.g., neuropathic pain or inflammatory pain.

Agents are obtained from a wide variety of sources including libraries of synthetic or natural compounds.

A variety of other reagents can be included in the screening assays to enhance or optimize assay conditions. These include reagents like salts, neutral proteins, e.g., albumin, detergents, etc., which can be used to facilitate optimal protein-protein binding and/or reduce non-specific or background interactions. Also, reagents that otherwise improve the efficiency of the assay, such as protease inhibitors, nuclease inhibitors, anti-microbial agents, and the like may be used.

Alternatively, the GLT-1 protein is used to generate a crystal structure. Once the three-dimensional structure is determined, a potential agonistic agent can be examined through the use of computer modeling using a docking program such as GRAM, DOCK, or AUTODOCK (Dunbrack, et al. 1997. Folding & Design 2:27-42). This procedure can include computer fitting of potential agents to GLT-1 to ascertain how well the shape and the chemical structure of the potential ligand will enhance glutamate transport. Computer programs can also be employed to estimate the attraction, repulsion, and steric hindrance of the agent. Generally the tighter the fit (e.g., the lower the steric hindrance, and/or the greater the attractive force) the more potent the potential agent will be since these properties are consistent with a tighter binding constraint. Furthermore, the more specificity that is employed in the design of a potential agent, the more likely it is that the agent will not interfere with related mammalian proteins. This will minimize potential side-effects due to unwanted interactions with other proteins.

Assays can be carried out in vitro utilizing a source of GLT-1 which is naturally isolated, or recombinantly produced GLT-1, in preparations ranging from crude to pure. Recombinant GLT-1 can be produced in a number of prokaryotic or eukaryotic expression systems which are well-known in the art. Such assays can be performed in an array format. In certain embodiments, one or a plurality of the assay steps are automated.

Assays can, in one embodiment, be performed using an appropriate host cell as a source of GLT-1. Such a host cell can be prepared by the introduction of DNA encoding GLT-1 into the host cell and providing conditions for the expression of GLT-1. Such host cells can be prokaryotic or eukaryotic, bacterial, yeast, amphibian or mammalian.

Nucleic acids encoding GLT-1 can be delivered to cells in vivo using methods such as direct injection of DNA, receptor-mediated DNA uptake, viral-mediated transfection or non-viral transfection and lipid based transfection. Direct injection has been used to introduce naked DNA into cells in vivo (see, e.g., Acsadi, et al. 1991. Nature 332:815-818; Wolff, et al. 1990. Science 247:1465-1468). A delivery apparatus (e.g., a gene gun) for injecting DNA into cells in vivo can be used. Such an apparatus is commercially available (e.g., from BIO-RAD). Naked DNA can also be introduced into cells by complexing the DNA to a cation, such as polylysine, which is coupled to a ligand for a cell-surface receptor (see, for example, Wu and Wu. 1988J. Biol. Chem. 263:14621; Wilson, et al. 1992 J. Biol. Chem. 267:963-967; and U.S. Pat. No. 5,166,320). Binding of the DNA-ligand complex to the receptor can facilitate uptake of the DNA by receptor-mediated endocytosis. A DNA-ligand complex linked to adenovirus capsids which disrupt endosomes, thereby releasing material into the cytoplasm, can be used to avoid degradation of the complex by intracellular lysosomes (see, for example, Curiel, et al. 1991. Proc. Natl. Acad. Sci. USA 88:8850; Cristiano, et al. 1993. Proc. Natl. Acad. Sci. USA 90:2122-2126).

As the skilled artisan can appreciate, any of the above cell-based and cell-free assays can be adapted for use with other components of the GLT-1 signalling pathway to identify agents which ultimately modulate the expression or activity of GLT-1 and are therefore useful for preventing or treating acute and chronic pain, e.g., neuropathic pain or inflammatory pain. While agents that modulate the growth factor-dependent expression of GLT-1 could be of uses it is contemplated that activators of the GLT-1 signalling pathway that is dependent upon cAMP are of particular use in preventing or treating acute and chronic pain. Examples of the cAMP-dependent signalling pathway components leading to GLT-1 expression include, but are not limited to, PKA, MEK, and PI3K. In this regard, Zelenaia and colleagues (2000. Mol. Pharmacol. 57: 667-678) teach that a PKA inhibitor, e.g., KT5720, blocks induction of GLT-1 protein in db-cAMP-treated astrocytes. Further, PD98059, a selective inhibitor of MEK (Dudley, et al. 1995. Proc. Natl. Acad. Sci. USA 92:7686-7689) partially attenuates the effects of db-cAMP-dependent GLT-1 expression (Zelenaia, et al. 2000. Mol. Pharmacol. 57: 667-678), indicating that the MEK-MAP/Erk pathway may be involved in regulation of GLT-1 expression by cAMP. Moreover, LY294002, a specific and potent inhibitor of phosphatidylinositol 3-kinase (PI3K; Duronio, et al. 1998. ell Signal 10:233-239), blocks induction of GLT-1 protein in db-cAMP-treated cultures (Zelenaia, et al. 2000. Mol. Pharmacol. 57: 667-678). This study further found that downstream mediators of the PI3K-dependent signaling are also involved in cAMP-dependent expression of GLT-1. For example, PDTC, an inhibitor of nuclear transcription factor-κB (NF-κB) activation, inhibited db-cAMP-mediated increases in GLT-1 expression. As such, agents which increase the expression or activity of PKA (e.g., PKA agonists Sₚ-cAMP-S or 8Br-cAMP), MEK, PI3K, or NF-κB or activate the synthesis of cAMP (e.g., forskolin) can also be screened in accordance with the instant assays and used in the prevention and treatment of acute and chronic pain, e.g., neuropathic pain or inflammatory pain. The read-out of such assays can be based upon the expression or activity of the individual GLT-1 signaling pathway component being assayed or can be determined based upon the expression or activity of GLT-1.

Agents identified by the screening assays disclosed herein are also embraced by the present invention. To demonstrate efficacy in the prevention and treatment of acute and chronic pain, e.g., neuropathic pain or inflammatory pain, it is desirable that the instant agents be tested in, for example, the animal model described herein.

The invention is described in greater detail by the following non-limiting examples.

### EXAMPLES

### Example 1

### Primary Astrocyte Culture

Astrocyte cultures were prepared from the cortices of neonatal rats (1-3 days old) using the Worthington Papain Dissociation System (Worthington Biochemical Corporation, Lakewood, N.J.) according to the method of Huettner and Baughman (1986. J. Neurosci. 6:3044-3060). Briefly, cortices of neonatal rats were dissected, treated with papain (20 U/ml), dissociated by trituration and plated in 75 cm² flasks in Dulbecco's modified Eagle's medium (DMEM) supplemented with 10% charcoal-stripped fetal bovine serum (FBS), 1% GlutaMax and 1% penicillin/streptomycin (P/S, 100 U/ml penicillin, 100 μg/ml streptomycin, P/S). Cells were fed twice weekly until they reached confluence (Day in vitro, DIV 10-12) at which point they were mechanically shaken for 1 hr on an orbital shaker to remove any remaining oligodendrocytes and microglia. Subsequently, cultures were treated with trypsin for 30 minutes at 37° C. and re-plated into 6 or 12-well dishes at a density of about 3.0×10⁴ cells/cm². Resultant cultures were greater than 95% astrocytic as determined by GFAP immunostaining.

### Example 2

### Treatment of Cultured Astrocytes

After approximately 14 days (2 days after subplating into plates, DIV 14) astrocytes had formed a confluent monolayer. The culture medium was exchanged and replaced with fresh DMEM containing 10% FBS, 1% GlutaMax and 1% P/S. Saline, propentofylline (10, 100 or 1000 μM), dibutyryl cyclic-adenosine-5′,3′-monophosphate (db-cAMP, 125 or 250 μM) or lipopolysaccharide (1 μg/ml) were added and cells were incubated for 1, 3 or 7 days further at 37° C. Phase contrast microscopy was then carried out in order to assess the morphology of cells after the various treatments. Length of astrocyte processes was quantified by measuring all processes in 2-3 phase contrast images/group by an independent observer, blinded to the treatment groups using Image J v1.34s (National Institutes of Health, Bethesda, Md.).

### Example 3

### Real Time RT-PCR

Total RNA was isolated from astrocyte cultures using the Qiagen RNeasy mini-kit (Qiagen Inc., Valencia, Calif.) according to the manufacturer's protocol for isolation of total RNA from animal cells. Reverse transcription (RT) was carried out using the high-capacity cDNA archive kit (Applied Biosystems, Foster City, Calif.) according to the vendor's protocol. Real-time RT-PCR reactions were carried out in a total reaction volume of 25 μL containing a final concentration of 1.5 U Platinum Taq DNA polymerase (Invitrogen Corporation, Carlsbad, Calif.); 20 mM Tris HCl (pH 8.4); 50 mM KCl; 3 mM MgCl₂; 200 μM dGTP, dCTP, and dATP; 400 μM dUTP and 1 U of UDG (uracyl DNA glycosylase); 900 nM of forward and reverse primers; 300 nM Taqman probe; and 5 μL of cDNA (50 ng) from the RT step. Primer and probe sequences for the genes of interest (GLT-1, GLAST and GAPDH) are shown in Table 1. The iCycler™ Multicolor Real-Time PCR detection system (Bio-Rad Laboratories, Hercules, Calif.) was used to quantify PCR product. The fluorescence and threshold values (C_{T}) obtained were used to compare the relative amount of target mRNA in experimental groups to those of controls using the 2^{-ΔΔC}^{_{T}} method (Livak, K. J. and T. D. Schmittgen. 2001. Methods 25:402-408). Each experiment was run twice and samples were run in duplicate. For each sample, the mean C_{T} value for the control gene (GAPDH) was then subtracted from the mean C_{T} value for the gene of interest (GLT-1, GLAST) to obtain a ΔC_{T} value. The ΔC_{T} values for the control group (untreated) were then averaged and subtracted from the ΔC_{T} for the experimental groups to obtain the ΔΔC_{T}. The relative fold change from control was then expressed by calculation of 2^{-ΔΔC}^{_{T}} for each sample and the results are expressed as the group mean fold change±SEM.

TABLE 1

| | Accession | | | SEQ ID |
|---|---|---|---|---|
| Gene | Number | Primers/Probes | Sequence* | NO: |
| GADPH | NM_01008 | Forward primer | 5′-CCCCCAATGTATCCGTTGTG-3′ | 1 |
| | | Reverse primer | 5′-TAGCCCAGGATGCCCTTTAGT-3′ | 2 |
| | | TAQMAN probe | 5′-TGCCGCCTGGAGAAACCTGCC-3′ | 3 |
| GLT-1 | X67857 | Forward primer | 5′-GAGCATTGGTGCAGCCAGTATT-3′ | 4 |
| | | Reverse primer | 5′-GTTCTCATTCTATCCAGCAGCCAG-3′ | 5 |
| | | TAQMAN probe | 5′-CAGCGCCGGGTTGGTCACCA-3′ | 6 |
| GLAST | X63744 | Forward primer | 5′-CCTGGGTTTTCATTGGAGGG-3′ | 7 |
| | | Reverse primer | 5′-ATGCGTTTGTCCACACCATTG-3′ | 8 |
| | | TAQMAN probe | 5′-TGCCACCCTGCCCATCACTTTCAA-3′ | 9 |

*The TAQMAN probe has a reporter fluorescent dye, FAM (6-carboxyfluorescein) at the 5′ end and fluorescence dye quencher, TAMRA (6-carboxytetramethyl-rhodamine) at the 3′ end.

### Example 4

### Immunochemistry

Primary astrocyte-enriched cultures were plated onto sterile 18 mm glass coverslips. After three washes in PBS, cells were permeabilized in 5% glacial acetic acid/95% ethanol (acid-alcohol) for 10 minutes. After washing, cells were incubated in a 1% normal goat serum for 30 minutes and then overnight at 4° C. in primary mouse anti-GFAP (1:500), rabbit anti-GLT-1 (1:500) or guinea pig anti-GLAST (1:2000). The following day, cells were washed and then incubated for 2 hours at room temperature with goat anti-mouse Alexa Fluor™-555 (Invitrogen Corporation, Carlsbad, Calif.) goat anti-guinea pig Alexa Fluor™-488 (Invitrogen Corporation, Carlsbad, Calif.) or goat anti-rabbit Alexa Fluor™-488 (Invitrogen Corporation, Carlsbad, Calif.; all at 1:250). Finally, cells were post-fixed in acid-alcohol and mounted with Vectashield (Vector Labs, Burlingame, Calif.) containing 4′,6-diamidino-2-phenylindole dihydrochloride hydrate (DAPI, Sigma Chemical Co., St Louis, Mo.), in order to visualize cell nuclei. The sections were examined with a fluorescence microscope, and images were captured with a Q-Fire cooled camera (Olympus, Melville, N.Y.). Confocal microscopy was also performed using a Zeiss LSM 510 Meta confocal microscope (Carl Zeiss AG, Oberkochen, Germany). Merged color images were processed using Adobe Photoshop 7.0 (Adobe Systems, San Jose, Calif.).

### Example 5

### Western Blot Analysis

Cells were scraped into PBS containing a protease inhibitor cocktail (1:1000, Sigma Chemical Co., St. Louis, Mo.) and then lysed by sonication in 5 one-second pulses. The samples were stored at −80° C. until use. Total protein was determined using the Lowry method (DC assay, Bio-Rad, Hercules, Calif.). Forty micrograms of protein (diluted in sample buffer and boiled) and standard protein markers were subjected to SDS/7.5% polyacrylamide gel electrophoresis and transferred to polyvinylidene difluoride (PVDF) membranes. Nonspecific binding was blocked by incubation with 5% milk/PBS-Tween at room temperature for 1 hour followed by incubation overnight at 4° C. with rabbit anti-GLT-1 (1:500) or guinea pig anti-GLAST (1:2500). The following day, blots were washed and incubated for 1 hour at room temperature with goat anti-rabbit HRP-conjugated secondary (1:3000) or goat anti-guinea pig HRP-conjugated secondary antibody (1:5000) and visualized with enhanced chemiluminescence (ECL). Images were obtained using the Typhoon Imaging System (Amersham-GE Healthcare, Piscataway, N.J.). Finally, blots were incubated for 15 minutes in stripping buffer and reprobed with a monoclonal mouse anti-β-actin antibody (1:10,000) as a loading control. Densitometric analysis was performed using ImageQuant 5.2 (Molecular Dynamics, GE Healthcare, Piscataway, N.J.).

### Example 6

### Measurement of Na+-Dependent Glutamate Uptake

Primary astrocytes were plated in 6-well plates, as described above. After 7 days of treatment with 10, 100 or 1000 μM propentofylline or db-cAMP, cells were washed twice in tissue buffer (0.05 M Tris/0.32 M Sucrose, pH 7.4). Where indicated, 100 μM dihydrokainate (DHK) was added to wells for 10 minutes before the addition of the substrate. Subsequently, [³H]-glutamate in Na⁺ Krebs buffer or Na⁺-free Krebs buffer (choline buffer) was added for 4 minutes at 37° C. The reaction was stopped by washing three times with ice cold buffer (0.05 M Tris/0.16 M NaCl, pH 7.4), and cells were lysed with 1 ml 0.1 N NaOH. The radioactivity in a 500 μl lysate aliquot was determined by liquid scintillation counting and a fraction of the lysate was also used for determination of protein concentration using the Lowry method (DC assay, Bio-Rad, Hercules, Calif.). Na⁺-dependent transport was calculated as the difference in the radioactivity accumulated in the presence and absence of Na⁺ and calculated as nmol/mg protein/min. Results are expressed as uptake relative to control samples.

### Example 7

### Enzyme-Linked Immunosorbent Assay (ELISA)

Standard ELISA was performed for quantitative determination of MCP-1 and MIP-2 protein in cell culture supernatant. Assays were carried out according to the manufacturer's specifications (Biosource, Camarillo, Calif.) using the sandwich enzyme immunoassay procedure. Optical density at 450 nm was obtained using the MRX Revelations program (Dynex Technologies, Chantilly, Va.) and relative protein concentrations were determined by comparing samples to the standard curve generated.

### Example 8

### Determination of cAMP Levels

For the determination of cAMP (or db-cAMP) in treated astrocytes, cells were washed twice with PBS plus 4 mM EDTA and 2 mM 3-isobutyl-1-methylxanthine (IBMX, Sigma Chemical Co., St. Louis, Mo.) and incubated for 15 minutes. Subsequently, cells were harvested and boiled for 3 minutes followed by high-speed (8,000×g) centrifugation. Fifty microliters of the resultant supernatant (total of 300 μl) was used to determine cAMP levels in the competition assay. cAMP levels were measured using a radio-receptor competition assay. In brief, [³H] cAMP was used in competition for a cAMP binding protein (PKA) against known concentrations of non-radiolabeled cAMP, followed by determination of the unknowns. The reaction was allowed to proceed for 2 hours at 4° C. Charcoal was used to remove excess unbound cAMP. Finally, samples were counted in 5 ml Liquiscint™ (National Diagnostics, Atlanta, Ga.). It should be noted that the assay will detect db-cAMP itself as this compound will also bind PKA.

### Example 9

### L5 Spinal Nerve Transection Surgery

Male Sprague Dawley rats (Harlan, Indianapolis, Ind.) weighing 150-175 g at the start of surgery were housed individually and maintained in a 12:12 light/dark cycle with ad libitum access to food and water. The animals were allowed to habituate to the housing facilities for 1 week before experiments began. Behavioral studies were performed in a quiet room between the hours of 7:00 and 10:00 A.M. The Institutional Animal Care and Use Committee at Dartmouth College approved all procedures in this study. Efforts were made to limit animal distress and use the minimum number of animals necessary to achieve statistical significance, in accord with guidelines set forth by the International Association for the Study of Pain.

Unilateral mononeuropathy was produced according to the method described by Colburn et al. (1999. Exp. Neurol. 157:289-304). Briefly, rats were anesthetized by inhalation of halothane in an O₂ carrier (induction, 4%; maintenance, 2%). A small incision to the skin overlying L5-S1 was made, followed by retraction of the paravertebral musculature from the superior articular and transverse processes. The L6 transverse process was partially removed, exposing the L4 and L5 spinal nerves. The L5 spinal nerve was identified, separated, lifted, and transected, followed by removal of a 3-mm distal segment of nerve to prevent reconnection. The wound was irrigated with saline and closed in two layers with 3-0 polyester suture (fascial plane) and surgical skin staples.

Mechanical sensitivity to non-noxious stimuli was measured by applying 2 g and 12 g von Frey filaments (Stoelting, Wood Dale, Ill.) to the plantar surface of the ipsilateral hind paw (n=8-12/group). Each round of testing consisted of 3 sets of 10 stimulations, with sets separated by 10 minutes from the previous (to avoid sensitization), for a total of 30 stimulations with each filament. The number of paw withdrawals observed is expressed out of a maximum of 30 possible withdrawals.

### Example 10

### Immunohistochemical Analysis in Rats

For assessment of GFAP, GLT-1 and GLAST immunoreactivity, a separate group of rats (n=4/treatment) were anesthetized and transcardially perfused with 0.1 M PBS (phosphate-buffered saline), pH 7.4, followed by 4% paraformaldehyde in PBS on day 12 post-L5 spinal nerve transection. Lumbar spinal cord sections were identified, isolated, and processed as described previously (Colburn, R. W. et al. 1997. J. Neuroimmunol. 79:163-175). Free-floating, 20 μM were cut in a cryostat and processed for immunofluorescence. Sections were blocked with 5% FBS/0.1% Triton-X 100 for 1 hour at room temperature (RT). For double immunofluorescence, spinal sections were incubated with a mixture of rabbit polyclonal anti-GLT-1 (1:500) or guinea pig anti-GLAST (1:5000, Chemicon, Temecula, Calif.), and mouse anti-GFAP G-A-5 (astrocyte marker, 1:400, Sigma Chemical Co., St. Louis, Mo.) in 3% FBS/0.1% triton-X 100/TBS over night at 4° C., washed, incubated in a mixture of goat anti-rabbit Alexa Fluor™-555 and goat anti-mouse Alexa Fluor™-488 (1:250, Molecular Probes, Eugene, Oreg.) secondary antibodies in 3% FBS/0.1% triton-X 100/TBS for 1 hour at RT. To control for nonspecific staining, control sections were incubated in the absence of primary antibodies. The sections were examined with a fluorescence microscope, and images were captured with a Q-Fire cooled camera. Merged color images were processed using Adobe Photoshop (Adobe Systems, San Jose, Calif.).

### Example 11

### Isolation of mRNA from Lumbar Spinal Cord

Tissue was collected from 4-5 rats/group on postoperative days 4 or 12. In order to obtain both mRNA and protein from the same tissue sample, the L5 region of the spinal cord (ipsilateral to the injury), was isolated and placed in PBS supplemented with protease inhibitor cocktail (1:1000, Sigma Chemical Co., St. Louis, Mo.) and RNase inhibitor (0.75 U/μL Ambion, Austin, Tex.). Samples were sonicated in five one-second bursts at half-maximal power, centrifuged at 6500 rpm for 15 minutes at 4° C. Protein-containing supernatants were collected and stored at −80° C. until further processing by Western blot analysis. The pellet was treated with TRIzol reagent (Invitrogen, Carlsbad, Calif.) for the extraction of total RNA according to the manufacturer's specifications.

### Example 12

### Real-Time PCR of Spinal Cord Tissue

Total RNA was isolated from 60-80 mg of lumbar spinal cord tissue on post-transection days 4 or 12 as described above. RNA samples were subsequently treated with DNAsel (DNA-Free Kit, Ambion, Austin, Tex.) to remove any contaminating genomic DNA. Reverse transcription (RT) was carried out using the high-capacity cDNA archive kit (Applied Biosystems, Foster City, Calif.) according to the vendor'S protocol.

Real time RT-PCR reactions were carried out in a total reaction volume of 25 μL containing a final concentration of 1.5 U Platinum Taq DNA polymerase (Invitrogen Corporation, Carlsbad, Calif.); 20 mM Tris HCl (pH 8.4); 50 mM KCl; 3 mM MgCl₂; 200 μM dGTP, dCTP, and dATP; 400 μM dUTP and 1 U of UDG (uracyl DNA glycosylase); 900 nM of forward and reverse primers; 300 nM Taqman probe; and 5 μL of a 10-fold dilution of cDNA (50 ng) from the RT step. Primer and probe sequences for the genes of interest (GLT-1, GLAST and GAPDH) are shown in Table 1. The iCycler™ Multicolor Real-Time PCR detection system (Bio-Rad, Hercules, Calif.) was used to quantify PCR product. The fluorescence and threshold values (C_{T}) obtained were used to compare the relative amount of target mRNA in experimental groups to those of controls using the 2^{-ΔΔC}^{_{T}} method (Livak and Schmittgen. 2001. Methods 25:402-408). Each experiment was run twice and samples were run in duplicate. For each sample, the mean C_{T} value for the control gene (GAPDH) was then subtracted from the mean C_{T} value for the gene of interest (GLT-1, GLAST) to obtain a ΔC_{T} value. The ΔC_{T} values for all animals in the control group (normal, s.c. saline) were then averaged and subtracted from the ΔC_{T} for each animal in the experimental groups to obtain the ΔΔC_{T}. The relative fold change from control was then expressed by calculation of 2^{-ΔΔC}^{_{T}} for each sample and the results are expressed as the group mean fold change±SEM.

### Example 13

### Western Blot Analysis of Spinal Cord

Protein obtained from L5 lumbar spinal cord was quantified using the Lowry method (DC assay, Bio-Rad, Hercules, Calif.). Forty micrograms of protein and standard protein markers were subjected to SDS polyacrylamide gel electrophoresis (7.5% gel, Bio-Rad, Hercules, Calif.) and id transferred to polyvinylidene difluoride (PVDF, Bio-Rad, Hercules, Calif.) filters. Nonspecific binding was blocked by incubation with 5% milk/PBS-T at room temperature for 1 hour followed by incubation overnight at 4° C. with monoclonal guinea pig anti-GLT-1 or guinea pig anti-GLAST (1:2500, Chemicon). The following day, blots were washed and incubated for 1 hour at room temperature with goat anti-guinea pig HRP-conjugated secondary antibody (1:10,000 for GLAST and 1:100,000 for GLT-1, Sigma Chemical Co., St. Louis, Mo.), visualized with SuperSignal West Femto Maximum Sensitivity Substrate (Pierce, Rockford, Ill.) for 1 minute and imaged using the Typhoon Imaging System (Amersham Biosciences, Piscataway, N.J.). Finally, blots were incubated for 15 minutes in stripping buffer and reprobed with a monoclonal mouse anti-β-actin antibody (1:10,000, Abcam, Cambridge, Mass.) as a loading control. Densitometric analysis was performed using ImageQuant 5.2 (Molecular Dynamics, Amersham Biosciences, Piscataway, N.J.).

### Example 14

### Transgenic Mouse Model

Transgenic mice on a C57B1/6 background were created according to the methods previously described (Regan et al. 2006. J. Neuroscience, in press). Briefly, in order to determine where in the central nervous system GLAST is expressed, transgenic mice were created using a bacterial artificial chromosome containing the GLAST gene plus 18 kb of DNA upstream of the first exon and 60 kb downstream of the last exon. DsRed cDNA was inserted into the first exon to allow expression of DsRed instead of GLAST when the promoter is active. Similarly, in order to investigate the distribution of GLT-1, transgenic mice expressing GLT-1 with eGFP running from the promoter were also created. These two types of transgenic mice were crossed and the resultant double transgenic eGFP-GLT-1/DsRed-GLAST mice were used in the experiments at 8 to 10 weeks of age.

### Example 15

### Immunohistochemical Analysis in Mice

As described previously with rats, mice were transcardially perfused with 0.1 M PBS (phosphate-buffered saline), pH 7.4, followed by 4%-paraformaldehyde in PBS on day 12 post-L5 spinal nerve transection. Lumbar spinal cord sections were identified, isolated, and processed as described previously (Colburn, R. W. et al. 1997. J. Neuroimmunol. 79:163-175). Free-floating, 20 μM were cut in a cryostat and processed for immunofluorescence. Sections were blocked with 5% FBS/0.1% Triton-X 100 for 1 hour at room temperature (RT). For double immunofluorescence, spinal sections were incubated with a mixture of rabbit polyclonal anti-GLT-1 (1:500) or guinea pig anti-GLAST (1:5000, Chemicon, Temecula, Calif.), and mouse anti-GFAP G-A-5 (astrocyte marker, 1:400, Sigma Chemical Co., St. Louis, Mo.) in 3% FBS/0.1% triton-X 100/TBS over night at 4° C., washed, incubated in a mixture of goat anti-rabbit Alexa Fluor™-555 and goat anti-mouse Alexa Fluor™-405 (1:250, Molecular Probes, Eugene, Oreg.) secondary antibodies in 3% FBS/0.1% triton-X 100/TBS for 1 hour at RT. To control for nonspecific staining, control sections were incubated in the absence of primary antibodies. The sections were examined with a fluorescence microscope, and images were captured with a Q-Fire cooled camera. Merged color images were processed using Adobe Photoshop (Adobe Systems, San Jose, Calif.). For both eGFP and DsRed, images were taken with a 397 ms exposure. For GFAP/Alexa 405, images were taken with a 131 ms exposure.

What is claimed is:

1. A method for preventing or treating acute and chronic pain in a patient comprising administering to a patient in need of treatment an effective amount of an agent that increases levels of glutamate transporter GLT-1, thereby preventing or treating acute and chronic pain in the patient.

2. The method of claim 1, wherein the effective amount comprises from 10 to 100 mg/m².

3. The method of claim 1, wherein the effective amount comprises from 20 to 90 mg/m².

4. The method of claim 1, wherein the effective amount comprises from 30 to 80 mg/m².

5. The method of claim 1, wherein the effective amount comprises from 40 to 70 mg/m².

6. The method of claim 1, wherein the effective amount comprises from 50 to 60 mg/m².

7. The method of claim 1, wherein the agent is propentofylline or a methylxanthine derivative.

8. The method of claim 1, wherein the effective amount is about 60 mg/m².

9. The method of claim 1 wherein the acute and chronic pain is neuropathic pain or inflammatory pain.

10. The method of claim 1, wherein the agent is administered orally, sublingually, subcutaneously, intramuscularly, intravenously, or transmucosally.

11. A method for identifying an agent useful in preventing or treating acute and chronic pain comprising contacting GLT-1 or a cell expressing GLT-1 with a test agent and measuring the activity or expression level of GLT-1 in the presence and absence of the test agent, wherein an increase in the measured activity or expression level of GLT-1 in the presence of the test agent as compared with the measured activity or expression level of GLT-1 in the absence of the test agent identifies an agent that prevents or treats acute and chronic pain.

12. The method of claim 11, wherein the acute and chronic pain is neuropathic pain or inflammatory pain.

13. An agent for prevention or treatment of acute and chronic pain, wherein said agent is identified by the method of claim 11.

14. The agent of claim 13 further comprising a pharmaceutically acceptable vehicle.


SEQUENCE LISTING (9 sequences)

SEQ ID NO: 1 - DNA, 20 bp, Artificial Sequence
  Synthetic oligonucleotide
cccccaatgtatccgttgtg

SEQ ID NO: 2 - DNA, 21 bp, Artificial Sequence
  Synthetic oligonucleotide
tagcccaggatgccctttagt

SEQ ID NO: 3 - DNA, 21 bp, Artificial Sequence
  Synthetic oligonucleotide
tgccgcctggagaaacctgcc

SEQ ID NO: 4 - DNA, 22 bp, Artificial Sequence
  Synthetic oligonucleotide
gagcattggtgcagccagtatt

SEQ ID NO: 5 - DNA, 24 bp, Artificial Sequence
  Synthetic oligonucleotide
gttctcattctatccagcagccag

SEQ ID NO: 6 - DNA, 20 bp, Artificial Sequence
  Synthetic oligonucleotide
cagcgccgggttggtcacca

SEQ ID NO: 7 - DNA, 20 bp, Artificial Sequence
  Synthetic oligonucleotide
cctgggttttcattggaggg

SEQ ID NO: 8 - DNA, 21 bp, Artificial Sequence
  Synthetic oligonucleotide
atgcgtttgtccacaccattg

SEQ ID NO: 9 - DNA, 24 bp, Artificial Sequence
  Synthetic oligonucleotide
tgccaccctgcccatcactttcaa